(19) United States
(12) Patent Application Publication
(10) Pub. No.: US 2014/0336439 A1
(43) Pub. Date: Nov. 13, 2014

(54) PORTABLE PHANTOM DEVICE

(71) Applicants: Young Hoon Ji, Seoul (KR); Hyun Do Huh, Bucheon-si (KR); Sang Hyoun Choi, Bucheon-si (KR); Hai Jo Jung, Namyangju-si (KR); Kum Bae Kim, Seoul (KR)

(72) Inventors: Young Hoon Ji, Seoul (KR); Hyun Do Huh, Bucheon-si (KR); Sang Hyoun Choi, Bucheon-si (KR); Hai Jo Jung, Namyangju-si (KR); Kum Bae Kim, Seoul (KR)

(73) Assignee: KOREA INSTITUTE OF RADIOLOGICAL & MEDICAL SCIENCES, Seoul (KR)

(21) Appl. No.: 14/361,287

(22) PCT Filed: Aug. 28, 2012
(86) PCT No.: PCT/KR2012/006852

§ 371 Date: May 28, 2014

(30) Foreign Application Priority Data
Nov. 28, 2011 (KR) 10-2011-0125209

(51) Int. Cl. A61N 5/10

(52) U.S. Cl. CPC A61N 5/1075; A61N 2005/1076
USPC 600/1

(57) ABSTRACT

A portable phantom device according to the present invention is a phantom device which is detachably installed in a bird cage that is coupled to an irradiator, and includes a body provided with an accommodation space having a cylindrical shape and a top of which is open so as to accommodate water, a horizontality adjustment maintaining portion which is detachably coupled to a side wall of the body; and a sensor holding portion which is installed on the side wall of the body so as not to interfere with the horizontality adjustment maintaining portion, so that a sensor for measuring the amount of radiation that is incident on the accommodation space may be inserted from the outside of the body and accommodated in the body (Representative drawing: FIG. 2)

### TECHNICAL FIELD

The present invention relates to a phantom device for radiation treatment for measuring the amount of radiation from a radiation treatment apparatus which acts on a treatment region.

### BACKGROUND ART

Radiation for treatment in medical radiation is applied to a tumor of a cancer patient so as to prevent cancer cells from propagating further and to kill the cancer cells, thereby reducing the patient's pain.

For example, such radiation treatment is performed in order to prevent a recurrence of cancer when there is a high possibility that cancer cells remain after surgery, and to maximize an anti-cancer medicine treatment and an anti-cancer effect in the case of an inoperable cancer, in the case where a radiation treatment is more effective than surgery, and in the case where there is an attempt to improve the quality of a patient by performing both surgery and radiation treatment.

The radiation treatment is performed using a medical device including a radiation generator such as a linear accelerator. The linear accelerator may output high-dose X-rays or electron rays and may precisely adjust output energy, and thus has been recently used as standard equipment in radiation treatment.

Incidentally, in radiation treatment, it is particularly important to control radiation energy to be output from a radiation treatment apparatus at appropriate levels. Since a maximum treatment effect may be obtained by performing irradiation with optimal energy corresponding to the state, size, or depth of a tumor, it is very important to control a linear accelerator to output radiation with optimal energy.

Thus, it is necessary to confirm in advance operation precision such as whether an accelerator operates normally before using a radiation treatment apparatus or whether radiation with necessary energy is output, particularly, by normal adjustment of the amount of radiation. In addition, regarding such measurement of the amount of radiation, it is preferable that the amount of radiation with which a specific region of a patient's internal body to be actually treated is irradiated be measured.

In the medical community, a so-called phantom is used for such a purpose. The phantom is a measurement device which is manufactured to be capable of radiation measurement in place of a human body. An example of the phantom is disclosed in Korean Patent Publication No. 2007-0118394.

Incidentally, phantom devices of the related art have been manufactured to be usable mainly in places where a radiation treatment apparatus is installed, for example, hospitals where a specific radiation treatment apparatus is installed. More specifically, the phantom devices of the related art have a size which is difficult to transport to the outside, and has a structure which may not be directly attached to a radiation treatment apparatus. Accordingly, the phantom device of the related art operates an irradiator in a state where the phantom device is installed on a bed on which a patient to be treated is to lie, and measures the amount of radiation corresponding to the patient to be treated.

As such, there is a disadvantage in that the phantom device of the related art has to be necessarily provided in a place where a radiation treatment apparatus is installed, due to its low portability. In addition, in the case where a different type of phantom device is provided in each hospital, when a patient changes from one hospital to another, the same phantom device may not be used. Accordingly, there is a problem in that the amount of radiation with which the patient is irradiated may be slightly different. In addition, since the phantom device of the related art may not be attached to an irradiator, there is a problem in that the position of the irradiator has to be adjusted in order to measure the amount of radiation.

### DETAILED DESCRIPTION OF THE INVENTION

### Technical Problem

The present invention is contrived in order to solve the above-described problems, and an objective thereof is to provide a phantom device which is detachably installed in an irradiator and is easily carried so that the amount of radiation with which a patient is to be irradiated may be effectively measured and so that the same phantom device may be used when a patient is treated in a different hospital.

### Technical Solution

According to an aspect of the present invention, there is provided a portable phantom device which is detachably installed in a bird cage that is coupled to an irradiator. The phantom device includes a body provided with an accommodation space having a cylindrical shape and the top of which is open so as to accommodate water, a horizontality adjustment maintaining portion which is detachably coupled to a side wall of the body, and a sensor holding portion which is installed on the side wall of the body so as not to interfere with the horizontality adjustment maintaining portion, so that a sensor for measuring the amount of radiation that is incident on the accommodation space may be inserted from the outside of the body and accommodated therein.

### Advantageous Effects

A portable phantom device according to the present invention is detachably installed in a bird cage of a radiation treatment apparatus and has a structure which may be easily carried. Thus, it is possible to easily install the phantom device in order to measure the amount of radiation and to reduce a measurement error of the amount of radiation which is used for treatment by using the same phantom device even when a patient is treated in a different hospital.

### DESCRIPTION OF THE DRAWINGS

FIG. 1 is a schematic perspective view of a portable phantom device according to an embodiment of the present invention;

FIG. 2 is a schematic exploded perspective view of a major region of the portable phantom device shown in FIG. 1;

FIG. 3 is a cross-sectional view taken along a line III-III shown in FIG. 1;

FIG. 4 is a cross-sectional view taken along a line IV-IV shown in FIG. 3; and

FIG. 5 is a diagram showing a state where the portable phantom device of FIG. 1 is installed in an irradiator.

### BEST MODE

Hereinafter, an embodiment of the present invention will be described in detail with reference to the accompanying drawings.

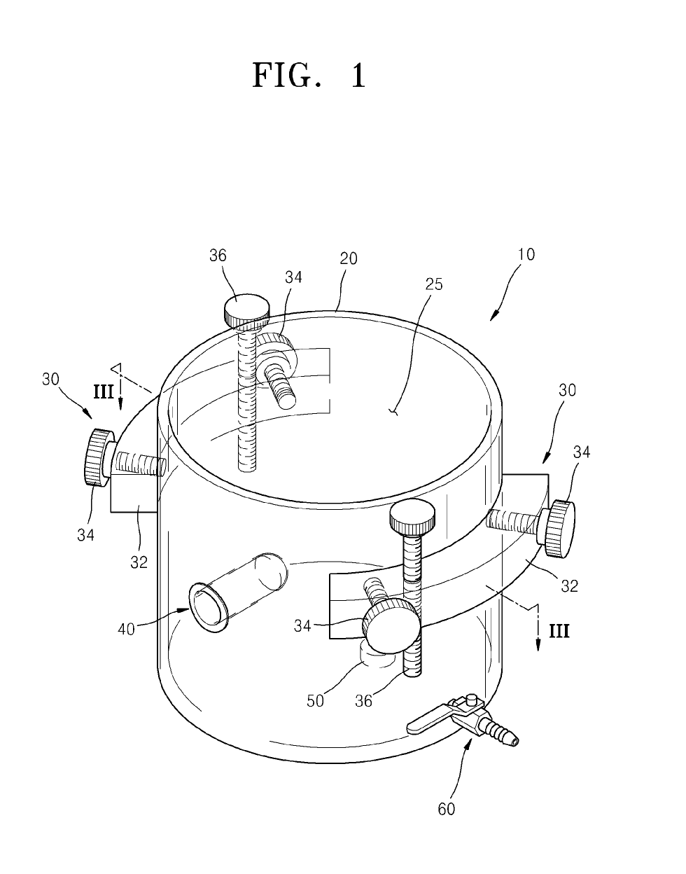

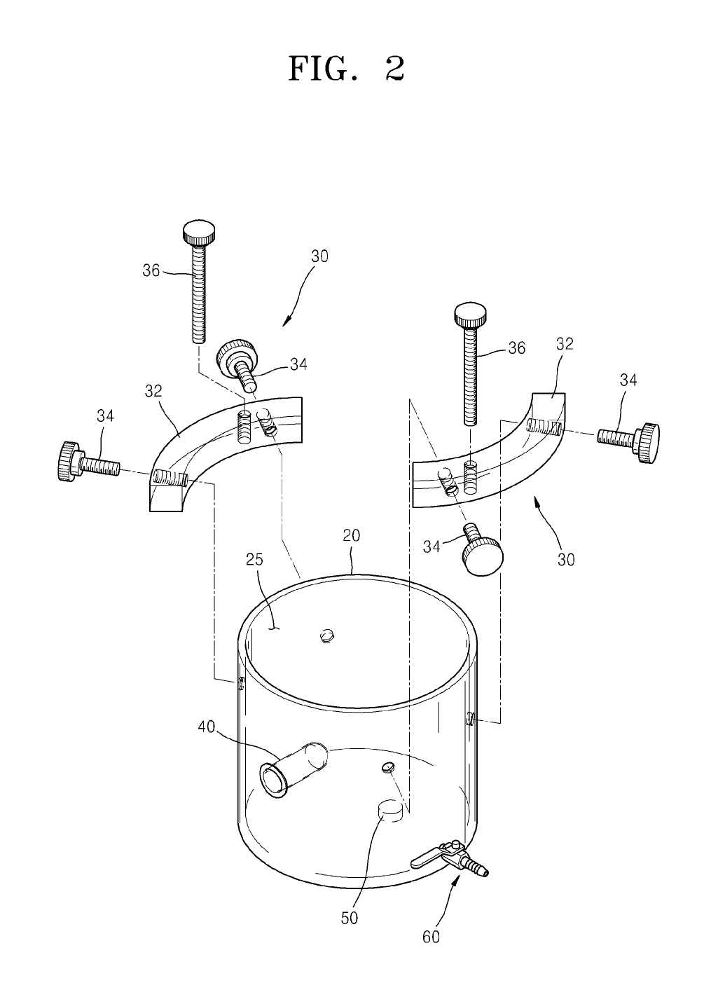

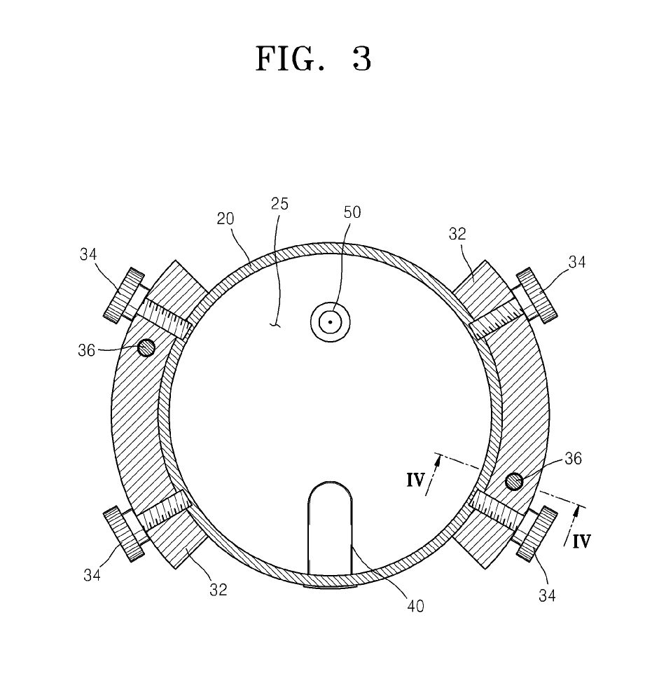

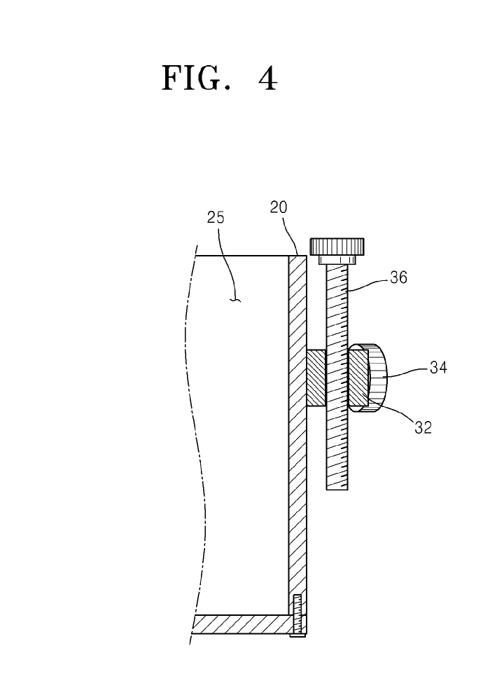

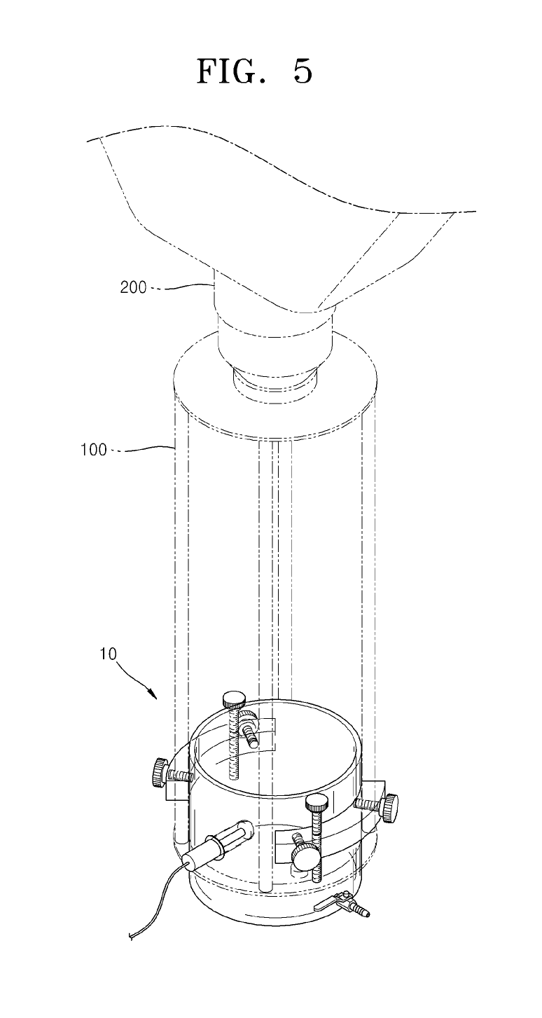

FIG. 1 is a schematic perspective view of a portable phantom device according to an embodiment of the present invention. FIG. 2 is a schematic exploded perspective view of a major region of the portable phantom device shown in FIG. 1. FIG. 3 is a cross-sectional view taken along a line III-III shown in FIG. 1. FIG. 4 is a cross-sectional view taken along a line IV-IV shown in FIG. 3. FIG. 5 is a diagram showing a state where the portable phantom device of FIG. 1 is installed in an irradiator.

Referring to FIGS. 1 to 5, a portable phantom device 10 (hereinafter, referred to as a “phantom device”) according to an embodiment of the present invention is a device for measuring the intensity of radiation irradiated from an irradiator.

The phantom device 10 is a device which is detachably installed in a bird cage 100 attached to an irradiator 200. In general, the irradiator 200 is provided with the bird cage 100 which is separately provided for the purpose of additionally installing necessary parts. The bird cage 100 may be detachably installed in the irradiator 200.

The phantom device 10 includes a body 20, a horizontality adjustment maintaining portion 30, a sensor holding portion 40, a leveler 50, and a drain valve 60.

The body 20 provides a space where a sensor for radiation measurement is installed. The body 20 has an accommodation space 25 capable of accommodating approximately two liters of water. The body 20 has a cylindrical shape of which a circular cross-section extends in a vertical direction. The bottom of the body 20 is closed, and the upper portion thereof is open. The body 20 is formed of a plastic material. In the current embodiment, the body 20 is formed of a transparent acrylic resin.

The side wall of the body 20 is provided with a plurality of grooves in which female screw portions are formed so that the horizontality adjustment maintaining portion 30 to be described later is installed. The groove having the female screw portion formed therein is formed to be concave inwards from the outside of the side wall and does not pass through the side wall. The lower portion of the side wall of the body 20 is provided with a hole for installing the drain valve 60 to be described later.

The horizontality adjustment maintaining portion 30 is detachably coupled to the side wall of the body 20.

The horizontality adjustment maintaining portion 30 is provided so as to maintain the horizontality of the body 20 and to be capable of adjusting a distance between the irradiator 200 and a sensor installed in the sensor holding portion 40 to be described later.

The horizontality adjustment maintaining portion 30 includes a supporting frame 32, a fixing member 34, and a horizontality adjustment member 36. All components constituting the horizontality adjustment maintaining portion 30 may be formed of a plastic material.

The supporting frame 32 is a member that extends in the circumferential direction of the side wall so as to come into surface contact with the side wall. The supporting frame 32 is provided with a plurality of holes in a direction horizontal to the ground. In the current embodiment, two holes are formed. In addition, the supporting frame 32 is provided with a vertical hole in a direction perpendicular to the ground. The vertical hole formed in the supporting frame 32 includes a female screw portion. In the current embodiment, two supporting frames 32 are provided.

The fixing member 34 is provided to detachably fix the supporting frame 32 to the side wall of the body 20. The fixing member 34 is a bolt-shaped member that has a head portion having a diameter larger than an inner diameter of the hold formed in the body 20. The fixing member 34 is threadably coupled to the groove formed in the outer side wall of the body 20 so as to fix the supporting frame 32 to the side wall of the body 20.

The horizontality adjustment member 36 is threadably coupled to the vertical hole formed in the supporting frame 32. The horizontality adjustment member 36 has a shape similar to that of the fixing member 34 and is formed to have a length greater than that of the fixing member 34. A lower portion of the horizontality adjustment member 36 is supported by the bird cage 100. The horizontality adjustment member 36, which is a bar-shaped member, has the lower portion supported by the bird cage 100 and an upper portion fixed to the supporting frame 32 so as to adjust the horizontality of the body 20. In the current embodiment, two horizontality adjustment members 36 are provided so as to be coupled to the respective supporting frames 32.

The sensor holding portion 40 is provided to protect the sensor, which is inserted into the accommodation space 25 of the body 20 for accommodating water, from the water. The sensor holding portion 40 is installed in the side wall of the body 20 so as not to interfere with the horizontality adjustment maintaining portion 30. The sensor holding portion 40 is provided so that the sensor for measuring the amount of radiation incident on the accommodation space 25 may be inserted into the sensor holding portion 40 from the outside of the body 20 and may be accommodated therein. One end of the sensor holding portion 40 is fixed in the hole formed in the side wall of the body 20, and the other end thereof protrudes toward the accommodation space 25 in the form of a pocket. The sensor holding portion 40 may be formed of a synthetic resin or rubber with flexibility.

The leveler 50 is provided on the bottom of the body 20. A bubble-type leveler which is ordinarily used may be adopted as the leveler 50. The leveler 50 is installed in an upper surface of the bottom of the body 20 by using a method such as bonding.

The drain valve 60 is installed in a lower portion of the side wall of the body 20. The drain valve 60 is provided in order to discharge water accommodated in the body 20.

Hereinafter, operations and effects of the present invention will be described in detail by using an example in which the portable phantom device 10 having the above-described structure is installed in the irradiator and removed therefrom.

The phantom device 10 is used to inspect in advance whether a patient's treatment region may be appropriately irradiated with radiation for treatment. There is an advantage in that the phantom device 10 has portability because it is manufactured to have a small size capable of accommodating approximately two liters of water. Accordingly, even when a patient who is usually treated in a certain hospital changes to a different hospital, the amount of radiation may be measured using the same phantom device, thereby obtaining an effect of removing a measurement error occurring when using a different phantom device. In a process of installing the phantom device 10 in the irradiator 200, first, the bird cage 100 is attached to the irradiator 200. As shown in FIG. 5, the bird cage 100 has an accommodation space having a cylindrical shape, includes a plurality of supports disposed in a vertical direction, and is provided with a circular frame in the lower portion thereof.

As shown in FIG. 2, the installation of the phantom device 10 is started in a state where the horizontality adjustment maintaining portion 30 is separated from the phantom device 10. The body 20 of the phantom device 10 is disposed at the inner lower portion of the bird cage 100. The supporting frame 32 is fixed to the body 20 by passing the fixing member 34 through the supporting frame 32 in a state where one hand grasps the body 20 and then by threadably coupling the fixing member 34 to the groove formed in the side wall of the body 20. The horizontality adjustment member 36 is threadably coupled to the supporting frame 32 in a direction perpendicular to the ground, and then the lower portion of the horizontality adjustment member 36 is supported by the bird cage 100.

The other supporting frame 32 is fixed to the body 20 in the same manner. The horizontality adjustment member 36 may adjust not only a distance between the phantom device 10 and the irradiator 200 but also the horizontality of the body 20. In this process, the horizontality of the body 20 may be precisely adjusted using the leveler 50. The accommodation space 25 is filled with water using the open surface of the top of the body 20 in a state where the drain valve 60 is closed. The sensor for radiation measurement is installed in the sensor holding portion 40 as shown in FIG. 5, and then the intensity of radiation irradiated from the irradiator 200 may be measured.

Meanwhile, a method of separating the phantom device 10 is as follows. First, the sensor installed in the sensor holding portion 40 is removed. Then, the drain valve 60 is opened so as to discharge the water accommodated in the accommodation space out of the body 20. The procedure of disassembling the horizontality adjustment maintaining portion 30 is performed in the reverse order to the above-described assembling order. In this manner, after the horizontality adjustment maintaining portion 30 is separated from the body 20, components constituting the horizontality adjustment maintaining portion 30 separated from the body 20 may be accommodated in the accommodation space 25 of the body 20. The phantom device 10 separated in this manner may be easily carried to a different place.

As described above, the phantom device 10 according to the present invention has a simple structure for installation and may easily adjust horizontality and a measurement position of a sensor, and thus the same phantom device may be used in a different place. As a result, even when a patient changes to a different hospital, the same phantom device is used so as to uniformly control the amount of radiation with which the patient is irradiated, thereby allowing a difference in the amount of radiation with which the patient is irradiated to be removed, as compared with a case where different phantom devices are used.

Although a preferred embodiment of the present invention has been described so far, the present invention is not limited to the above-described embodiment, and various types of embodiments may be set within the scope of the gist of the present invention.

### MODE OF THE INVENTION

In order to accomplish the above-described objective, an embodiment of the present invention provides a portable phantom device which is detachably installed in a bird cage attached to an irradiator. The portable phantom device includes a body which is provided with an accommodation space having a cylindrical shape and a top of which is open so as to accommodate water; a horizontality adjustment maintaining portion detachably coupled to a side wall of the body; and a sensor holding portion installed on the side wall of the body so as not to interfere with the horizontality adjustment maintaining portion, so that a sensor for measuring the amount of radiation that is incident on the accommodation space may be inserted from the outside of the body and accommodated in the body.

The horizontality adjustment maintaining portion may include a supporting frame extending in a circumferential direction of the side wall so as to come into surface contact with the side wall; a plurality of fixing members passing through the supporting frame so as to be threadably coupled to the side wall; and a horizontality adjustment member threadably coupled to the supporting frame and having a bar shape of which a lower portion is supported by the bird cage.

A bottom of the body may be provided with a leveler.

The body and the horizontality adjustment maintaining portion may be formed of a plastic material.

The sensor holding portion may be formed of a synthetic resin or rubber material having flexibility.

A lower portion of the side wall of the body may be provided with a drain valve so as to discharge water accommodated in the body.

1. A portable phantom device which is detachably installed in a bird cage attached to an irradiator, the portable phantom device comprising:
a body which is provided with an accommodation space having a cylindrical shape and a top of which is open so as to accommodate water;
a horizontality adjustment maintaining portion detachably coupled to a side wall of the body; and
a sensor holding portion installed on the side wall of the body so as not to interfere with the horizontality adjustment maintaining portion, so that a sensor for measuring the amount of radiation that is incident on the accommodation space may be inserted from the outside of the body and accommodated in the body.

2. The portable phantom device of claim 1, wherein the horizontality adjustment maintaining portion comprises
a supporting frame extending in a circumferential direction of the side wall so as to come into surface contact with the side wall;
a plurality of fixing members passing through the supporting frame so as to be threadably coupled to the side wall; and
a horizontality adjustment member threadably coupled to the supporting frame and having a bar shape of which a lower portion is supported by the bird cage.

3. The portable phantom device of claim 1, wherein a bottom of the body is provided with a leveler.

4. The portable phantom device of claim 1, wherein the body and the horizontality adjustment maintaining portion are formed of a plastic material.

5. The portable phantom device of claim 1, wherein the sensor holding portion is formed of a synthetic resin or rubber material having flexibility.

6. The portable phantom device of claim 1, wherein a lower portion of the side wall of the body is provided with a drain valve so as to discharge water accommodated in the body.

7. The portable phantom device of claim 2, wherein a bottom of the body is provided with a leveler.
